SEQUENCE LISTING (2 sequences)

SEQ ID NO: 1 - DNA, 70 bp, Artificial Sequence
  Description of Artificial Sequence Synthetic primer
atgaaacaaacacaacgtcacaacggtattatcgaactggttaaacagcagtgtaggctggagctgcttc

SEQ ID NO: 2 - DNA, 70 bp, Artificial Sequence
  Description of Artificial Sequence Synthetic primer
tgctgatgctgcccatattgaccatcgcgttacggccaaatttcgagtgacatatgaatatcctccttag

(19) United States
(12) Patent Application Publication
(10) Pub. No.: US 2009/0325243 A1
(43) Pub. Date: Dec. 31, 2009

(54) Method for producing amino acids using glycerol

(76) Inventors: Young Hoon Park, Gyeonggi-do (KR); Kwang Myung Cho, Gyeonggi-do (KR); Yong Uk Shin, Gyeonggi-do (KR); Hyun Ae Bae, Busan (KR); Jin Sook Chang, Seoul (KR); Jae Yeong Ju, Gyeonggi-do (KR)

Correspondence Address: EDWARDS ANGELL PALMER & DODGE LLP, P.O. BOX 55874, BOSTON, MA, 02205, US

(21) Appl. No.: 12/308,810

(22) PCT Filed: Jun. 26, 2007
(86) PCT No.: PCT/KR2007/003082

§ 371 Date: Jan. 29, 2009

(30) Foreign Application Priority Data
Jun. 26, 2006 (KR) 10-2006-0057633
Jun. 22, 2007 (KR) 10-2007-0061841

(51) Int. Cl. C12P 13/04; C12P 13/08; C12P 13/12; C12N 1/20

(52) U.S. Cl. 435/113; 435/106; 435/115; 435/252.8

(57) ABSTRACT

The present invention relates to an amino acid-producing microorganism capable of simultaneously utilizing glycerol as a carbon source, a method for preparing the microorganism, and a method for producing amino acids using the microorganism. According to the present invention, amino acids can be efficiently produced using a byproduct of biodiesel production, glycerol, thereby substituting a cheaper material for the conventional fermentation materials such as glucose.

### TECHNICAL FIELD

The present invention relates to an amino acid-producing microorganism capable of simultaneously utilizing glycerol as a carbon source, a method for preparing the microorganism, and a method for producing amino acids using the microorganism.

### BACKGROUND ART

Recently, in order to solve problems such as high oil prices due to an increase in the consumption of natural resources including petroleum, and environmental pollution (caused by the use thereof, much attention has been paid to the development of energy alternatives by using renewable materials in nature. Among them, ethanol obtained by fermentation (Bioethanol) and biodiesel obtained from oil derived from plants are highly considered as one of energy alternatives.

Biodiesel refers to fatty acid methyl ester or fatty acid ethyl ester, which is synthesized by esterification of oil derived from plants as a substrate with methanol in the presence of a catalyst. In this process, 10% by weight of glycerol is inevitably produced as a byproduct, based on the total weight.

Glycerol (C₃H₈O₃) is chemically more reduced than glucose (C₆H₁₂O₆), thus providing a higher reducing power for metabolism of a microorganism. Since a lot of materials produced during fermentation are generally required to have a reducing power in their metabolism, the use of glycerol as a substrate can lead to significant improvement in yield and productivity. However, in spite of the properties, studies on glycerol are still limited to reuterin (Talarico et. al., Antimicrob. Agents Chemother., 32:1854-1858 (1988)), 2,3-butanediol (Biebl, et al., Appl Microbiol. Biotechnol. 50:24-29 (1998)), 1,3-propanediol (Menzel, et. al., Enzyme Microb. Technol., 20:82-86 (1997)), succinic acid (Korean Patent No. 0313134), Itaconic acid (U.S. Pat. No. 5,457,040), 3-hydroxypropanaldehyde (Doleyres et al. Appl. Micribiol. Biotechnol. 68(4):467-474 (2005)), and propionic acid (Himmi et al., Appl. Microbiol. Biotechnol., 53: 435-440 (2000)). There is a reason that glycerol is more expensive than other carbon sources effectively used in the conventional fermentation industry. On the contrary, methods for producing glycerol by fermentation have been studied (Wang et al., Biotechnol. Adv., 19(3): 201-223 (2001)). However, with the dramatic increase in biodiesel production, glycerol production has increased, which has lead to a decrease in its price. Based on the above facts, there is a report that the byproducts of biodiesel production including glycerol are employed in the production of 1,3-propanediol (Gonzalez-Pajuelo et al., J. Ind. Microbiol. Biotechnol. 31: 442-446, (2004)), and hydrogen and ethanol (Ito et al., J. Biosci. Bioeng., 100(3): 260-265 (2005)). However, a method for producing amino acids and major metabolites as a representative fermentation product using the byproducts of biodiesel production including glycerol has not yet been reported.

Until now, glycerol has been obtained from the manufacturing process of soaps, fatty acids, waxes, surfactants, or the like. However, as described above, with the dramatic increase in biodiesel production, glycerol production will also increase as its byproduct, thereby generating a problem of effectively treating the byproducts including glycerol. Further, the price of refined glycerol is expected to decrease sharply. Accordingly, a production of useful chemical materials by fermentation using glycerol can provide a lot of additional effects.

The glycerol metabolism in microorganisms has been better understood in Escherichia coli and Klebsiella pneumoniae. In Escherichia coli, the aquaglyceroporin GlpF facilitates the uptake of glycerol from the environment without requiring energy consumption (Heller et al., J. Bacteriol. 144:274-278, (1980)). The glycerol is converted to glycerol-3-phosphate by a glycerol kinase (GlpK), next converted to dihydroxyacetonephosphate (DHAP) by a glycerol-3-phosphate dehydrogenase, and then converted to glyceraldehyde-3-phosphate (G-3-P) by a triosephosphate isomerase (TpiA), so as to be metabolized through glycolysis (Lin E C, Annu. Rev. Microbiol. 30:535-578, (1976)). In the case where the glycerol kinase has no activity, glycerol is converted to dihydroxyacetone (DHA) by a glycerol dehydrogenase (Gdh), next converted to dihydroxyacetone phosphate (DHAP) by glycerol kinase or dihydroxyacetone kinase (DHA kinase), and then converted to glyceraldehyde-3-phosphate (G-3-P), so as to be metabolized (Paulsen et al., Microbiology, 146: 2343-2344, (2000)). The glycerol metabolism is regulated in various ways. In particular, in the presence of glycerol with glucose, Escherichia coli wild-type has been known to show diauxic growth such that glucose is preferentially used before glycerol (Lin, Annu. Rev. Microbiol. 30:535-578, (1976)).

As mentioned above, when glycerol obtained as a byproduct of biodiesel production is effectively used as a carbon source, a lot of value-added can be obtained. Further, in the presence of glycerol with glucose as carbon source, Escherichia coli wild-type shows diauxic growth such that glucose is preferentially used before glycerol. Therefore, when a complex carbon source containing glycerol is supplied, fermentation efficiency is reduced. Based on the facts, the present inventors have made extensive studies on glycerol utilization by microorganisms, thereby completing the present invention.

### DISCLOSURE OF INVENTION

### Technical Problem

It is an object of the present invention to provide a method for producing amino acids with high efficiency and at low cost, in which an amino acid-producing microorganism capable of simultaneously utilizing glycerol as a carbon source is cultured in media containing glycerol.

It is another object of the present invention to provide an amino acid-producing microorganism capable of simultaneously utilizing glycerol as the carbon source, and a method for preparing the microorganism.

### BEST MODE FOR CARRYING OUT THE INVENTION

In one embodiment, the present invention provides a method for producing amino acids using glycerol, comprising the steps of inoculating and culturing an amino acid-producing microorganism capable of simultaneously utilizing glycerol as a carbon source in culture media containing glycerol, and recovering amino acids from the media obtained in the above step.

As used herein, the phrase “amino acid-producing microorganism capable of simultaneously utilizing glycerol as a carbon source” refers to a microorganism having an ability of producing amino acids using other carbon sources than glycerol, and simultaneously producing amino acids using glycerol as a carbon source. Other carbon sources than glycerol are a carbon source known in the related art, for example, carbohydrates such as sucrose, fructose, lactose, glucose, maltose, starch, and cellulose, fats such as soybean oil, sunflower oil, castor oil, and coconut oil, fatty acids such as palmitic acid, stearic acid, and linoleic acid, preferably glucose, fructose, and lactose, more preferably glucose. The microorganism of the invention is able to produce amino acids simultaneously using the above carbon sources and glycerol as a carbon source, thereby having higher efficiency of producing final amino acids, as compared to a microorganism preferentially utilizing the above carbon source and then utilizing glycerol. Specifically, in the case of simultaneously supplying glucose and glycerol as a carbon source, diauxic growth is observed, in which wild-type Escherichia coli exclusively utilizes glucose and exhausts it, and then utilizes glycerol. Therefore, fermentation efficiency is reduced, in the case of supplying complex carbon sources containing glycerol. On the contrary, an amino acid-producing microorganism capable of simultaneously utilizing glycerol as a carbon source is different from the wild-type strain, in that it can simultaneously utilize glucose and glycerol in the presence of both glucose and glycerol, rather than in the presence of glucose or glycerol, so as to increase fermentation efficiency, thereby producing a larger amount of amino acids.

The microorganism of the invention preferably has a galR gene and/or glpR gene in its genome, and any one or both of the genes may be inactivated. A GalR protein produced by the expression of the galR gene has been known to inhibit the expression of a gene encoding GalP protein, which is a permease that transports a variety of sugars including galactose and glucose into a cell (MARK GEANACOPOULOS AND SANKAR ADHYA, Journal of Bacteriology, January 1997, p. 228-234, Vol. 179, No. 1). It has been known that a GlpR protein produced by the expression of the glpR gene is a regulatory factor of glycerol-3-phosphate metabolism, and binds to an operator of glpD, glpFK, glpTQ, and glpABC operons involved in glycerol metabolism, and inhibits the transcription of the genes (Larson et al., J. Bio. Chem. 262(33): 15869-15874; Larson et al., J. Biol. Chem. 267(9): 6114-6121 (1992); Zeng et al., J. Bacteriol. 178(24): 7080-7089, (1996)). The present inventors have found that the efficiency of glycerol utilization can be improved by increasing the GalP protein expression or by inactivating a representative regulatory factor of glycerol metabolism, glpR, thereby trying to inactivate the related genes. Examples of the inactivation method include a method comprising the steps of inducing mutation using radiation such as ultra-violet or chemicals, and screening the strains having the inactivated glpR gene and/or galR gene from the obtained mutants, and any method known to those skilled in the art can be employed. Further, the inactivation method includes a method using DNA recombination technology. The DNA recombination technology can be done by introducing a nucleotide sequence or vector containing a nucleotide sequence having homology with glpR gene and/or galR gene into the microorganism, so as to generate homologous recombination. Further, the nucleotide sequence or vector to be introduced may contain a dominant selective marker. The sequence of the glpR gene and galR gene are disclosed, and can be obtained from a database such as National Center for Biotechnology Information (NCBI) and the DNA Data Bank of Japan. Further, the glpR gene and galR gene in Escherichia coli are disclosed, and can be obtained from the genome sequence of Escherichia coli disclosed by Blattner et. al. (Science 277:1453-1462(1997). Further, the glpR gene and galR gene include alleles that are caused by degeneration or silent mutation at a codon. As used herein, the term “inactivation” means that the active glpR gene and/or galR gene are not expressed, or the expression of the glycerol metabolism-related genes is not inhibited, or an active GalP is not expressed. Therefore, if the glpR gene is inactivated, the expression of the glycerol metabolism-related genes or a combination thereof is increased, and if the galR gene is inactivated, the GalP expression is increased.

In the present invention, the microorganism is a microorganism capable of producing amino acids, and a microorganism simultaneously utilizing glycerol, preferably including the galR gene and/or glpR gene in its genome, and any microorganism including any one or both of the genes inactivated is not limited to prokaryotes or eukaryotes. Examples of the microorganism include a microorganism-belonging to the genus Escherichia, Enterobacteria, Brevibacterium, Corynebacterium, Klebsiella, Citrobacter, Streptomyces, Bacillus, Lactobacillus, Pseudomonas, Saccharomyces, and Aspergillus, preferably a microorganism belonging to the family Enterobacteriaceae, more preferably a microorganism belonging to the genus Escherichia, even more preferably Escherichia coli, most preferably Escherichia coli FTR2537 and FrR2533 (KCCM-10540 and KCCM-10541) (Korean Patent Publication No. 2005-0079344), Escherichia coli CJM002(KCCM-10568), Escherichia coli CJIT6007 (KCCM-10755P), and Escherichia coli derived therefrom. The microorganisms can simultaneously utilize glycerol as a carbon source. As a result, they have better ability of producing amino acids in the case of supplying glycerol rather than in the case of not supplying glycerol as a carbon source.

In the microorganisms, a high L-threonine-producing strain, Escherichia coli FTR2533 is derived from Escherichia coli FTR7624 by inactivating the galR gene (Korean Patent Publication No. 2005-0079344), and the Escherichia coli FTR7624 is derived from KCCM-10236. The Escherichia coli FTR7624 is a strain capable of increasing the production amount of L-threonine, by inactivating a tyrR gene in the genome of KCCM-10236. KCCM-10236 is a strain capable of increasing the production amount of L-threonine, in which the strain is resistant to L-threonine analogs, isoleucine leaky auxotrophic, resistant to L-lysine analogs, and resistant to α-aminobutyric acid, and a phosphoenolpyruvate carboxylase gene (ppc) and genes involved in threonine synthetic pathway (thrA: aspartokinase 1-homoserine dehydrognase, thrB: homoserine kinase, thrC: threonine synthase) are introduced (Korean Patent Publication No. 2005-0079344). Further, a high L-methionine-producing strain, Escherichia coli CJM002 (KCCM-10568) is derived from a parent strain, Escherichia coli FTR2533, in which L-methionine auxotrophicity of the parent strain was removed by NTG mutation. The Escherichia coli CJIT6007 is a strain that has both of the inactivated glpR and galR gene, in which a deletion cassette containing polynucleotide sequence having homology with glpR was prepared by PCR, and then introduced into the Escherichia coli FTR2533 strain.

In the method for producing amino acids using the microorganism of the invention, the process of culturing the microorganism can be performed according to suitable media and culture conditions known in the art. Those skilled in the art can easily modify the culture process depending on the selected strain. Examples of the culture method include batch culture, continuous culture, and fed-batch culture methods, but are not limited thereto. The various culture methods are disclosed, for example, in [“Biochemical Engineering”, James M. Lee, Prentice-Hall International Editions, pp 138-176].

The media used in the culture method should preferably meet the requirements of a specific strain. The media used in the present invention partially or totally contains glycerol as a carbon source, and may contain a suitable amount of other carbon sources. The carbon sources are well known to those skilled in the art, for example, carbohydrates such as sucrose, fructose, lactose, glucose, maltose, starch, and cellulose, fats such as soybean oil, sunflower oil, castor oil, and coconut oil, and fatty acids such as palmitic acid, stearic acid, and linoleic acid. The culture media preferably contains 1 g to 300 g of glycerol per liter. In the media, the glycerol content is 10 to 100% by weight, based on the total weight of carbon source, and if the content is out of the range, the production yield of amino acid is reduced. In addition to the carbon sources, examples of nitrogen source capable of being used include an organic nitrogen source such as peptone, yeast extract, meat extract, malt extract, corn steep liquor, and soy meal, and an inorganic nitrogen source such as urea, ammonium sulfate, ammonium chloride, ammonium phosphate, ammonium carbonate, and ammonium nitrate, and they can be used singly or in any combination thereof. As a phosphorus source, the media may contain potassium dihydrogen phosphate, dipotassium hydrogen phosphate, and corresponding sodium-containing salts. Further, the media may contain metal salts such as magnesium sulfate and iron sulfate. In addition, the media may contain amino acids, vitamins, and suitable precursors. The media or precursors can be added in batch culture, or continuous culture. Compounds such as ammonium hydroxide, potassium hydroxide, ammonia, phosphoric acid, and sulfuric Acid are added to the media during culture, so as to adjust the pH of the media. Further, during culture, an anti-foaming agent such as fatty acid polyglycol ester is used to inhibit the formation of foam. Further, in order to maintain the aerobic condition of the culture media, oxygen or oxygen-containing gas can be injected into the culture media. In order to maintain anaerobic and microaerobic conditions, nitrogen, hydrogen, or carbon dioxide is injected without injection of gas. Temperature of the culture media is generally 20° C. to 45° C., preferably 25° C. to 40° C. The culture period is a period of continuously producing amino acids, preferably 10 to 160 hours.

In order to recover amino acids from the culture media, a method known in the art can be used, and ion-exchange chromatography or the like can be employed, but ate not limited thereto.

Examples of the amino acids produced by the method of the present invention include industrially useful aspartate, threonine, lysine, methionine, isoleucine, asparagine, glutamic acid, glutamine, proline, alanine, valine, leucine, tryptophan, tyrosine, phenylalanine, serine, glycine, cysteine, arginine, and histidine, but are not limited thereto, preferably aspartate, lysine, threonine, and methionine, more preferably threonine and methionine.

In one embodiment, the present invention relates to an amino acid-producing microorganism simultaneously utilizing glycerol as a carbon source. In a specific embodiment, the present invention relates to an amino acid-producing microorganism simultaneously utilizing glycerol as a carbon source, in which the microorganism has the inactivated glpR gene and/or galR gene in its genome.

The microorganism of the invention is a microorganism capable of producing amino acids, which simultaneously utilizes glycerol, preferably any microorganism having the galR gene and/or glpR gene in its genome, in which any one or both of the genes are inactivated, are not limited to prokaryotic microorganism and eukaryotic microorganism. Examples of the microorganism include microorganisms belonging to the genus Escherichia, Enterobacteria, Brevibacterium, Corynebacterium, Klebsiella, Citrobacter, Streptomyces, Bacillus, Lactobacillus, Pseudomonas, Saccharomyces, and Aspergillus, preferably microorganisms belonging to the family Enterobacteriaceae, more preferably microorganisms belonging to the genus Escherichia, even more preferably Escherichia coli, and most preferably Escherichia coli CJIT6007 (Deposit No. KCCM-10755P).

In another embodiment, the present invention relates to a method for preparing the amino acid-producing microorganism simultaneously utilizing glycerol as a carbon source, in particular, the amino acid-producing microorganism simultaneously utilizing glycerol and having the inactivated galR gene and/or glpR gene.

In one specific embodiment, the present invention relates to a method for preparing the microorganism that can efficiently utilize glycerol, comprising the steps of preparing the inactivated glpR gene or a DNA fragment thereof; introducing the gene or the DNA fragment thereof into the microorganism capable of producing amino acids, to recombine with the glpR gene in its genome; and screening the microorganism, in which the glpR gene is inactivated.

In the method of the present invention, the microorganism preferably belongs to the family Enterobacteriaceae, and the microorganism is more preferably Escherichia coli, and most preferably Escherichia coli CJIT6007 (Deposit No. KCCM-10577P).

In the method of the present invention, the inactivated glpR gene or the DNA fragment thereof refers to a polynucleotide sequence, in which the polynucleotide sequence contains a polynucleotide sequence having sequence homology with the glpR gene in host, and mutation such as deletion, substitution, and inversion is introduced into the sequence, so as not to express the active glpR gene product. The procedure of introducing the inactivated glpR gene or the fragment thereof into a host cell can be preformed by transformation, conjugation, transduction, or electroporation, but are not limited thereto.

In the case of introducing the inactivated glpR gene or the DNA fragment thereof into a host cell by transformation, the inactivation can be performed by mixing the polynucleotide sequence with the culture media of the strain. At this time, the strain is naturally competent to accept DNA, thus being transformed. However, it is preferable that the strain had been made competent by a suitable method for DNA influx. The inactivated glpR gene or the DNA fragment thereof introduces a foreign DNA fragment into a fragment of the genome DNA, and substitutes a wild-type copy of this sequence with an inactivated form. In one specific embodiment, the inactivated polynucleotide sequence contains a tail including a portion of the target-site DNA in 5′ and 3′-terminal regions. For convenience, the inactivated polynucleotide sequence may contain a selectable marker, for example, an antibiotic-resistance gene. In the case where the target DNA is inactivated by the antibiotic-resistance gene, the selection of transformants is performed on an agarose plate containing a suitable antibiotic. The inactivated polynucleotide sequence introduced into a host cell by transformation can inactivate the wild-type genome sequence by homologous recombination with a tail sequence of the genome DNA.

In another specific embodiment, the present invention relates to a method for preparing a microorganism, in which any one or both of galR gene and glpR gene sequentially or simultaneously is/are inactivated by the same method as described above.

In one example of the method of the present invention, the method for preparing the microorganism, in which the glpR gene that regulates the glycerol metabolism related genes is inactivated, in order to effectively produce amino acids using various carbon sources including glycerol by fermentation, comprises the following process.

First, the deletion cassette containing the polynucleotide sequence having homology with the glpR gene is prepared using a pKD3 plasmid as a template by PCR. Next, Escherichia coli containing a pKD46 plasmid with a recombinase gene is transformed with the DNA fragment obtained from the PCR. The transformed Escherichia coli is plated on an agar plate containing an antibiotic marker, and then the strains having antibiotic-resistance are screened to isolate the strain having the inactivated glpR gene.

In one specific example of the invention, the present inventors prepared the deletion cassette containing the polynucleotide sequence having homology with the glpR gene by PCR, and then introduced it into a high L-threonine-producing strain, Escherichia coli FTR2533. As a result, they have developed a new strain, in which the wild-type glpR gene is inactivated, so as to utilize glycerol more efficiently than the parent strain, and to produce L-threonine with high yield. The new strain was designated as Escherichia coli CJIT6007, and deposited in Korean Culture Center of Microorganisms under the Budapest Treaty on Jun. 2. 2006 (Deposit No. KCCM-10755P).

Hereinafter, the present invention will be described in detail with reference to Examples. However, these Examples are for illustrative purposes only, and the invention is not intended to be limited by these Examples.

### MODE FOR THE INVENTION

### Example

### Example 1

### Flask Test for Simultaneous Utilization of Glycerol by Threonine-Producing Strain (Glucose and Glycerol)

Escherichia coli wild-type strain, K12 and FTR2533 strains were each inoculated in plates containing MMYE, and cultured at 33° C. incubator for 12 hours. Then, each strain was inoculated with the aid of a platinum loop in MMYE liquid media, and cultured at 33° C. and 200 rpm for 6 hours. The composition of MMYE media is shown in the following Table 1.

TABLE 1
Composition of MMYE medium

| | | |
|---|---|---|
| Glucose | 2 | g |
| MgSO₄•7H₂O | 0.493 | g |
| CaCl₂ | 0.011 | g |
| Na₂HPO₄•12H₂O | 6 | g |
| NaCl | 0.5 | g |
| KH₂PO₄ | 3 | g |
| Yeast extract | 10 | g |

| | |
|---|---|
| DW | Added to be 1 L |

Glucose, CaCl₂, and MgSO₄.7H₂O were separately sterilized. Before sterilizing the media, 2.2□ of 4N KOH was added thereto. Each 500□ of K12 and FTR2533 strains cultured in MMYE were inoculated in 25□ of titer media (250□ volume flask), and cultured at 33° C. and 200 rpm for 48 hours. The consumption patterns of carbon source depending on time were observed by using each strain in threonine titer media containing different ratios of glucose to glycerol. As a result, it was confirmed whether glycerol was simultaneously utilized by the FTR2533 strain or not. The composition of threonine titer media is shown in the following Table 2.

TABLE 2
Composition of threonine titer medium

| | | |
|---|---|---|
| C-source | 70 | g |
| KH₂PO₄ | 2 | g |
| (NH₄)₂SO₄ | 25 | g |
| MgSO₄•7H₂O | 1 | g |
| MnSO₄•4H₂O | 0.01 | g |
| FeSO₄•7H₂O | 0.01 | g |
| DL-Met | 0.15 | g |
| Yeast extract | 2 | g |
| CaCO₃ | 30 | g |

| | |
|---|---|
| DW | Added to be 1 L |

C-source and KH₂PO₄ were separately sterilized, and 2.2□ of 4N KOH was added thereto, before sterilizing the media. The C-source was prepared with five different ratios of glucose to glycerol, as shown in the following Table 3.

TABLE 3

| | Glucose-Glycerol |
|---|---|
| 1 | 70-0 |
| 2 | 52.5-17.5 |
| 3 | 35.0-35.0 |
| 4 | 17.5-52.5 |
| 5 | 0-70 |

(Unit; g/L)

The media was diluted 500 times with distilled water, and centrifuged to obtain supernatant. Then, HPLC was performed to analyze the production amount of threonine. The media was diluted 10 times with distilled water, and centrifuged to obtain supernatant. Then, HPLC was performed to analyze the production amount of glucose and glycerol.

An optical density (OD) was measured at 562 nm with media, which had been diluted 50 times with 0.3 N HCl solution. The following Tables 4 and 5 show the results of flask test for wild-type strain K12 and threonine producing strain FTR2533, respectively. The remaining amount of glucose and glycerol, and the production amount of threonine were compared at 12 hours, 24 hours, and 48 hours after starting the experiments. Glc represents glucose, Gly represents glycerol, and Thr represents threonine. Each unit is g/L.

TABLE 4
Result of flask titer test for wild-type strain K12

| | 12 hr | | | | 24 hr | | | | 48 hr | | | |
|---|---|---|---|---|---|---|---|---|---|---|---|---|
| Glucose:Glycerol | OD | Glc | Gly | Thr | OD | Glc | Gly | Thr | OD | Glc | Gly | Thr |
| 70:0 | 18.2 | 49 | 0 | 0 | 21.9 | 33.3 | 0 | 0 | 21.0 | 17.2 | 0 | 0 |
| 52.5:17.5 | 17.6 | 33.2 | 17.4 | 0 | 21.0 | 21.8 | 17.3 | 0 | 20.3 | 12.2 | 17.0 | 0 |
| 35:35 | 14.1 | 14.4 | 35.0 | 0 | 20.1 | 5.5 | 34.5 | 0 | 19.0 | 0 | 35.0 | 0 |
| 17.5:52.5 | 13.4 | 0 | 51.3 | 0 | 20.5 | 0 | 20.7 | 0 | 20.0 | 0 | 15.8 | 0 |
| 0:70 | 15.8 | 0 | 49.9 | 0 | 22.6 | 0 | 36.3 | 0 | 21.8 | 0 | 19.0 | 0 |

TABLE 5
Result of flask titer test for threonine producing strain FTR2533

| | 12 hr | | | | 24 hr | | | | 48 hr | | | |
|---|---|---|---|---|---|---|---|---|---|---|---|---|
| Glucose:Glycerol | OD | Glc | Gly | Thr | OD | Glc | Gly | Thr | OD | Glc | Gly | Thr |
| 70:0 | 4.2 | 69.2 | 0 | 1.7 | 18.8 | 38.3 | 0 | 8.8 | 19.2 | 0 | 0 | 23.1 |
| 52.5:17.5 | 4.8 | 52.5 | 15.7 | 2.2 | 19.2 | 29.9 | 3.5 | 11.4 | 22.7 | 0 | 0 | 23.9 |
| 35:35 | 4.8 | 32.3 | 33.6 | 2.0 | 20.5 | 15.7 | 21.5 | 12.1 | 19.9 | 0 | 0 | 26.7 |
| 17.5:52.5 | 4.3 | 14.0 | 52.5 | 1.8 | 19.9 | 0 | 36.2 | 10.5 | 20.6 | 0 | 0 | 24.4 |
| 0:70 | 4.3 | 0 | 68.5 | 1.6 | 12.4 | 0 | 47.4 | 7.9 | 19.6 | 0 | 0 | 28.4 |

As shown in Tables 4 and 5, the wild-type strain K12 preferentially consumed glucose in complex titer media containing glycerol and glucose at 12 hours and 24 hours after starting the flask cultivation, and then consumed glycerol. As a result, it was found that the wild-type strain K12 did not simultaneously utilize glycerol. However, it was found that the threonine producing strain FTR2533 simultaneously utilized glycerol and glucose from 12 hours after starting cultivation (Table 5). Furthermore, it was found that the threonine producing strain FTR2533 produced threonine 15% more in the complex media containing 50% glycerol as a carbon source, and 23% more in the media containing only glycerol, than in the media containing only glucose as a carbon source. The FTR2533 strain was found to produce threonine with high yield in the media containing glycerol (Table 5).

### Example 2

### Preparation of Recombinant Plasmid and Inactivation of glpR Gene Using the Same (Knock-Out)

In this Example, a glpR gene in the genome of Escherichia coli was inactivated by homologous recombination. For this, an FRT-one-step PCR deletion method was used (PNAS, 97: 6640-6645 (2000)). PCR was performed using primers represented by SEQ ID NOs. 1 and 2, and a pKD3 vector as a template (PNAS, 97: 6640-6645 (2000)), so as to prepare a deletion cassette. The PCR steps of denaturation, annealing, and extension were performed at 94° C. for 30 seconds, at 55° C. for 30 seconds, and at 72° C. for 1 minute, respectively. The cycle was repeated 30 times.

Forward Primer:

(SEQ ID NO. 1)

5′ ATGAAACAAACACAACGTCACAACGGTATTATCGAACTGGT-
TAAACAGCAGTGTAGGCTGGAGCTGCTTC 3′

Reverse Primer:

(SEQ ID NO. 2)

5′ TGCTGATGCTGCCCATATTGACCATCGCGTTACGGCCAAATTTCGAG
T-GACATATGAATATCCTCCTTAG 3′

Electrophoresis was performed with the obtained PCR product on a 1.0% agarose gel, and DNA was isolated from the 1.2 Kb size of band.

The recovered DNA fragment was electrophorated into the Escherichia coli FTR2533 strain, which had been transformed with a pKD46 vector (PNAS, 97:6640-6645 (2000)). For the electrophoration, the FTR2533 strain containing the pKD46 vector was cultured in LB media containing 100 □/L ampicillin and 5 mM L-arabinose at 30° C. to be an OD₆₀₀ of 0.6. Then, the strain was washed with sterilized water twice, and washed with 10% glycerol once for use. The electrophoration was performed at 2500 V. The recovered strain was plated on LB solid media containing 25 □/L chloramphenicol, and cultured at 37° C. overnight. Then, the strain showing antibiotic-resistance was screened. PCR was performed using the screened strain as a template and the same primers under the same conditions. In order to confirm the deletion of glpR gene, the amplified DNA was run on a 1.0% agarose gel, so as to confirm whether its size is 1.2 Kb. The confirmed strain was transformed with a pCP20 vector (PNAS, 97: 6640-6645 (2000)), and cultured in LB media. Then, PCR was performed under the same conditions, and run on the 1.0% agarose gel to confirm its size of 150 bp. The size reduction indicates that the chloramphenicol maker was removed. Finally, an FTR2533ΔglpR strain, in which the glpR genie was deleted, was obtained. The prepared strain was designated as CJIT6007.

### Example 3

### Production of L-Threonine by CJIT6007 Strain

Escherichia coli CJIT6007, in which a glycerol metabolism regulatory factor glpR had been deleted, was used to confirm the simultaneous utilization of glycerol and productivity of L-threonine in the complex media containing glycerol as a carbon source, and in the threonine titer media containing only glycerol.

The CJIT6007 strain was inoculated in MMYE plates, and cultured at 33° C. incubator for 12 hours. Then, the strain was inoculated with the aid of a platinum loop in MMYE liquid media, and cultured at 33° C. and 200 rpm for 6 hours. The composition of MMYE media is as shown in Table 1. Glucose, CaCl₂, and MgSO₄.7H₂O were separately sterilized. Before sterilizing the media, 2.2□ of 4N KOH was added thereto.

500□ of the strain cultured in MMYE were inoculated in 25□ of titer media (Table 2), and cultured at 33° C. and 200 rpm for 48 hours. The initial concentration of glycerol was 70 g/L. Glycerol and KH₂PO₄ were separately sterilized, and 2.2□ of 4 N KOH was added thereto, before sterilizing the media The media was diluted 500 times with distilled water, and centrifuged to obtain supernatant. Then, HPLC was performed to analyze the production amount of threonine. The media was diluted 10 times with distilled water, and centrifuged to obtain supernatant. Then, HPLC was performed to analyze the production amount of glucose and glycerol. An optical density (OD) was measured at 562 nm with media, which had been diluted 50 times with 0.3 N HCl solution. The results are shown in Table 6. Table 6 shows the results of flask test for the threonine producing strain. The remaining amount of glucose and glycerol, and the production amount of threonine were confirmed at 12 hours, 24 hours, and 48 hours after starting cultivation. Gly represents glycerol, and Thr represents threonine. Each unit is g/L.

TABLE 6
Result of flask titer test for threonine producing strain FTR2533ΔglpR

| | 12 hr | | | | 24 hr | | | | 48 hr | | | |
|---|---|---|---|---|---|---|---|---|---|---|---|---|
| Glucose:Glycerol | OD | Glc | Gly | Thr | OD | Glc | Gly | Thr | OD | Glc | Gly | Thr |
| 70:0 | 5.3 | 67.3 | 0 | 2.0 | 17.5 | 34.5 | 0 | 8.1 | 20.1 | 0 | 0 | 23.4 |
| 52.5:17.5 | 5.5 | 50.8 | 14.6 | 2.4 | 18.9 | 29.8 | 0.9 | 11.5 | 20.5 | 0 | 0 | 25.2 |
| 35:35 | 5.5 | 30.2 | 32.8 | 2.2 | 19.8 | 9.9 | 21.5 | 12.8 | 21.4 | 0 | 0 | 29.0 |
| 17.5:52.5 | 5.6 | 13.1 | 48.0 | 2.0 | 18.7 | 0 | 33.9 | 10.1 | 21.0 | 0 | 0 | 26.1 |
| 0-70 | 5.3 | 0 | 68.2 | 1.7 | 12.8 | 0 | 46.5 | 8.6 | 18.7 | 0 | 0 | 28.2 |

As shown in Table 6, it was found that the recombinant strain FTR2533ΔglpR, in which the glpR gene had been inactivated, simultaneously utilized glycerol and glucose in the complex titer media containing glucose and glycerol. Furthermore, the strain was found to have higher productivity of threonine than the FT1R2533 strain under the same condition. In particular, the strain was found to consume the carbon source with much higher rate in the complex media containing 50% glycerol as a carbon source (glucose:glycerol=35:35) at 24 hours after starting cultivation, as compared to the FTR2533 strain. Finally, the productivity of threonine by the FTR2533ΔglpR strain was found to be improved 8.6% more than that by the FTR2533 strain (Tables 5 and 6). Accordingly, it was found that the FTR2533ΔglpR strain, in which the glpR gene had been inactivated, simultaneously utilized glycerol as a carbon source to produce L-threonine with higher yield, as compared to the strain, in which the glpR gene had not been inactivated.

### Example 4

### Fermentation for Producing Methionine

A methionine producing strain, Escherichia coli CJM002 (KCCM-10568) described in PCT Publication NO. WO 06/001616 was used in complex media containing glycerol as a carbon source to perform methionine production test. The Escherichia coli CJM002, in which methionine biosynthetic pathway was accelerated, was obtained from a parent strain, Escherichia coli FTR2533. For methionine production test, the strain was cultured in Erlenmeyer flasks. The KCCM-10568 strain was plated on LB plates, and cultured at 31° C. overnight. Then, a single colony was inoculated in 3□ of LB media, and cultured at 31° C. for 5 hours. Then, the culture media was diluted 200 times in 250□ Erlenmeyer flask containing 25□ of methionine producing media, and cultured at 31° C. and 200 rpm for 64 hours. HPLC analysis was performed with the cultured strain to compare the production amount of methionine.

TABLE 7
Methionine producing medium

| | Concentration | | | | |
|---|---|---|---|---|---|
| | (per liter) | Medium | Medium | Medium | |
| Composition | Medium A | B | C | D | Medium E |
| Glucose | 40 g | 30 g | 20 g | 10 g | 0 g |
| Glycerol | 0 g | 10 g | 20 g | 30 g | 40 g |
| Ammonium | 17 g | 17 g | 17 g | 17 g | 17 g |
| sulfate | | | | | |
| KH₂PO₄ | 1.0 g | 1.0 g | 1.0 g | 1.0 g | 1.0 g |
| MgSO₄•7H₂O | 0.5 g | 0.5 g | 0.5 g | 0.5 g | 0.5 g |
| FeSO₄•7H₂O | 5 mg | 5 mg | 5 mg | 5 mg | 5 mg |
| MnSO₄•4H₂O | 5 mg | 5 mg | 5 mg | 5 mg | 5 mg |
| ZnSO₄ | 5 mg | 5 mg | 5 mg | 5 mg | 5 mg |
| Calcium | 30 g | 30 g | 30 g | 30 g | 30 g |
| carbonate | | | | | |
| Yeast extract | 2 g | 2 g | 2 g | 2 g | 2 g |
| pH (7.0) | | | | | |

TABLE 8
Productivity comparison of L-methionine

| | | Consumption of | Consumption of | L-methionine |
|---|---|---|---|---|
| Medium | OD | glucose (g/L) | glycerol (g/L) | (g/L) |
| A | 14.2 | 40 | 0 | 0.35 |
| B | 8.1 | 30 | 9 | 0.41 |
| C | 8.0 | 20 | 19.5 | 0.27 |
| D | 14.7 | 10 | 30 | 0.42 |
| E | 11.9 | 0 | 40 | 0.64 |

As shown in Table 8, the CJM002 strain was also found to effectively produce L-methionine using the complex media containing glucose and glycerol. In particular, 80% increase in the production yield of L-methionine was found in medium E containing only glycerol, as compared to medium A containing only glucose.

### INDUSTRIAL APPLICABILITY

According to the present invention, an amino acid-producing microorganism capable of simultaneously utilizing glycerol as a carbon source is used to efficiently produce amino acids in complex media containing a byproduct of biodiesel production, glycerol as a carbon source or in media containing only glycerol, thereby substituting a cheaper material for the conventional fermentation materials such as glucose.

1. A method for producing amino acids using glycerol, comprising the steps of:
inoculating and culturing an amino acid-producing microorganism capable of simultaneously utilizing glycerol as a carbon source in culture media containing glycerol; and
recovering amino acids from the culture media of the above step.

2. The method according to claim 1, wherein the glycerol is contained in content of 1 g to 300 g in 1 L of culture media.

3. The method according to claim 1, wherein the glycerol content is 10 to 100% by weight, based on the total weight of carbon source in the culture media.

4. The method according to claim 1, wherein the produced amino acids are threonine or methionine.

5. The method according to claim 1, wherein the amino acid-producing microorganism has an inactivated galR gene and/or glpR gene in its genome.

6. The method according to claim 1, wherein the microorganism belongs to the family Enterobacteriaceae.

7. The method according to claim 6, wherein the microorganism is Escherichia coli.

8. The method according to claim 7, wherein the Escherichia coli is KCCM-10540, KCCM-10541, KCCM-10568, or KCCM-10755P.

9. An amino acid-producing microorganism capable of simultaneously utilizing glycerol as a carbon source.

10. The microorganism according to claim 9, wherein the microorganism has an inactivated galR gene and/or glpR gene in its genome.

11. The microorganism according to claim 10, wherein the microorganism belongs to the family Enterobacteriaceae.

12. The microorganism according to claim 11, wherein the microorganism is Escherichia coli.

13. The microorganism according to claim 12, wherein the Escherichia coli is Escherichia coli KCCM-10540, KCCM-10541, KCCM-10568, or CJIT6007 (Deposit No. KCCM-10755P).

14. The microorganism according to claim 9, wherein the produced amino acids are threonine or methionine.

15. A method for preparing an amino acid-producing microorganism capable of simultaneously utilizing glycerol as a carbon source, comprising the steps of:
(a) preparing an inactivated glpR gene or a DNA fragment thereof;
(b) introducing the inactivated glpR gene or the DNA fragment thereof into a microorganism capable of producing amino acids, so as to recombine with the glpR gene in its genome; and
(c) screening the microorganisms having the inactivated glpR gene.

16. The method according to claim 15, wherein the microorganism of the step (b) further has an inactivated galR gene.

17. The method according to claim 14, wherein the microorganism belongs to the family Enterobacteriaceae.

18. The method according to claim 16, wherein the microorganism is Escherichia coli.

19. The method according to claim 17, wherein the microorganism is Escherichia coli CJIT6007 (Deposit No. KCCM-10755P).
